(19) United States
(12) Patent Application Publication
(10) Pub. No.: US 2024/0360060 A1
(43) Pub. Date: Oct. 31, 2024

(54) 6-HYDROXY-CANNABIDIOL-C4

(71) Applicant: GW RESEARCH LIMITED, Cambridgeshire (GB)

(72) Inventors: Alan James SILCOCK, Cambridge (GB); Karen TSE, Cambridge (GB)

(21) Appl. No.: 18/560,316

(22) PCT Filed: May 11, 2022
(86) PCT No.: PCT/GB2022/051198

§ 371 Date: Nov. 10, 2023

(30) Foreign Application Priority Data
May 12, 2021 (GB) 2106789.7

(51) Int. Cl. C07C 39/23; A61K 31/00; A61K 45/06; A61P 25/08

(52) U.S. Cl. CPC C07C 39/23; A61K 31/658; A61K 45/06; A61P 25/08

(57) ABSTRACT

The present invention relates to a compound that is pharmaceutically active and methods of preparation thereof. The compound of the invention is 6-hydroxy-cannabidol-C4 (6-OH-CBD-C4). The compound of the invention is related to cannabidiol (CBD). CBD is a non-psychoactive cannabinoid which has been used to treat various diseases and disorders. While such treatments hold promise, there remains a need in the art for more effective treatments and this has been brought about by way of the compound of the invention.

### RELATED APPLICATIONS

The present application is related to, and claims the benefit of, GB 2106789.7 filed on 12 May 2021 (12.05.2021), the contents of which are hereby incorporated by reference in their entirety.

### FIELD OF THE INVENTION

The present invention relates to a novel compound that is pharmaceutically active and methods of preparation thereof. In particular, the present invention relates to 6-hydroxy-cannabidol-C4 (6-OH-CBD-C4) and its use in the treatment of epilepsy.

### BACKGROUND TO THE INVENTION

Epilepsy occurs in approximately 1% of the population worldwide, (Thurman et at., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation (Epidiolex®) was approved by the FDA in June 2018 and the EMA in September 2019 for the treatment of seizures associated with Dravet and Lennox-Gastaut syndromes. In July 2020, the FDA approved the drug for the treatment of seizures associated with Tuberous Sclerosis Complex.

Cannabidiol-C4 (CBD-C4), also known as nor-cannabidiol is a homolog of CBD, with the side-chain shortened by one methylene bridge. It is a naturally occurring cannabinoid that can be found in minor quantities in the cannabis plant. The cannabinoid can alternatively be produced by synthetic means.

Previously, the applicant has shown that the compound CBD-C4 exhibits anti-convulsant activity in WO 2020/104796. Therapeutic efficacy was shown in a MES model of generalised seizure, whilst a range of toxicology screens proved acceptable toxicology of the compound.

The present invention has been devised in light of these considerations.

### BRIEF SUMMARY OF THE INVENTION

At its most general, the present invention relates to the surprising discovery that the novel compound 6-OH-CBD-C4 is biologically active and hence useful in the treatment of diseases. Such a novel compound may be administered by a wide variety of routes including but not limited to oral, transdermal, buccal, nasal, pulmonary, rectal or ocular. Such a compound may be used for the treatment or prevention of medical conditions such as epilepsy.

In a first aspect of the invention there is provided a compound of formula (I), or a salt thereof:

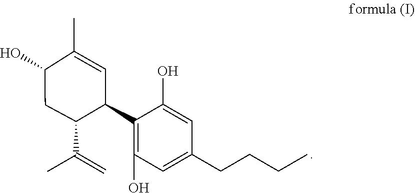 [SMILES: C=C(C)[C@@H]1C[C@H](O)C(C)=C[C@H]1c1c(O)cc(CCCC)cc1O]

In one embodiment, the compound of the first aspect is a pure, isolated or synthetic compound.

In a second aspect of the invention, there is provided a pharmaceutical composition comprising a compound of formula (I) or a salt thereof.

In one embodiment, the pharmaceutical composition of the second aspect comprises one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In one embodiment, the pharmaceutical composition of the second aspect is in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

In a third aspect of the invention there is provided a compound of formula (I) or a salt thereof, for use a medicament, such as a medicament for the treatment of epilepsy.

In a fourth aspect of the invention there is provided a compound of formula (I) or a salt thereof, for use in a method of treatment, such as a method of treating epilepsy.

In a fifth aspect of the invention there is provide a method of treatment comprising administering to a subject in need of treatment a therapeutically effective amount of a compound of formula (I) or a salt thereof.

In one embodiment, the compound of formula (I) or salt thereof is used in combination with one or more concomitant anti-epileptic drugs (AEDs).

In one embodiment, the dose of the compound of formula (I) is between 1 and 2,000 mg/kg.

In accordance with a sixth aspect of the present invention there is provided a process for the production of a compound of formula (I) comprising the following steps:
i) treating 4-butyl-5′-methyl-2′-(prop-1-en-2-yl)-1′,2′,3′,4′-tetrahydro-[1,1′-biphenyl]-2,6-diol with acetic anhydride to produce 4-Butyl-5′-methyl-2′-(prop-1-en-2-yl)-1′,2′,3′,4′-tetrahydro-[1,1′-biphenyl]-2,6-diyl diacetate;
ii) treating 4-Butyl-5′-methyl-2′-(prop-1-en-2-yl)-1′,2′,3′,4′-tetrahydro-[1,1′-biphenyl]-2,6-diyl diacetate with acetic anhydride and sodium dichromate dihydrate to produce 4-Butyl-5′-methyl-4′-oxo-2′-(prop-1-en-2-yl)-1′,2′,3′,4′-tetrahydro-[1,1′-biphenyl]-2,6-diyl diacetate; and
iii) treating 4-Butyl-5′-methyl-4′-oxo-2′-(prop-1-en-2-yl)-1′,2′,3′,4′-tetrahydro-[1,1′-biphenyl]-2,6-diyl diacetate with lithium aluminium hydride to produce the compound of Formula I.

In a seventh aspect of the invention there is provided an intermediate formed in the process of the production of a compound of formula (I), wherein the intermediate:

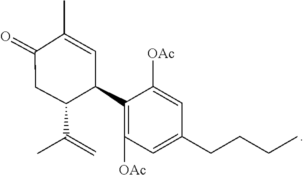 [SMILES: C=C(C)[C@@H]1CC(=O)C(C)=C[C@H]1c1c(OC(C)=O)cc(CCCC)cc1OC(C)=O]

These and other aspects and embodiments of the invention are described in further detail below.

### BRIEF SUMMARY OF THE DRAWINGS

There present invention is described with reference to the figures listed below:

FIG. 1 shows the evaluation of the test compound, as shown as compound I, in the mini-MEST test in the mouse as described in Example 2

### DETAILED DESCRIPTION OF THE INVENTION

The present invention relates to the compound 6-OH-CBD-C4, which is biologically active and hence useful in the treatment of diseases.

### Compound I

The invention provides a compound of formula (I):

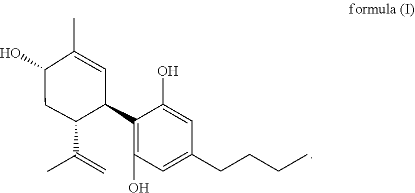 [SMILES: C=C(C)[C@@H]1C[C@H](O)C(C)=C[C@H]1c1c(O)cc(CCCC)cc1O]

The compound of formula (I) is (1′R,2′R,4′S)-4-butyl-5′-methyl-2′-(prop-1-en-2-yl)-1′,2′,3′,4′-tetrahydro-[1,1′-biphenyl]-2,4′,6-triol. It will be referred as the compound of formula (I) or compound I herein.

### Salts

In some embodiments, the compound of formula (I) is provided in free base form.

Alternatively, it may be convenient or desirable to prepare, purify, and/or handle a corresponding salt of the compound, for example, a pharmaceutically-acceptable salt.

Examples of pharmaceutically acceptable salts are discussed in “Pharmaceutical Salts: Properties, Selection, and Use”, 2^{nd} Edition, 2002, Stahl and Wermuth (Eds), Wiley-VCH, Weinheim, Germany.

Accordingly, in some embodiments the compound of formula (I) is provided as salts, for example in a protonated form together with a suitable counter anion.

Suitable counter anions include both organic and inorganic anions. Example of suitable inorganic anions include those derived from inorganic acids, including chloride (Cl⁻), bromide (Br⁻), iodide (I⁻), sulfate (SO₄²⁻), sulfite (SO₃²⁻), nitrate (NO₃⁻), nitrite (NO₂⁻), phosphate (PO₄³⁻), and phosphite (PO₃³⁻). Examples of suitable organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose.

Alternatively, in some embodiments the compound of formula (I) is provided as salts, for example in a deprotonated form together with a suitable counter cation.

Suitable counter cations include both organic and inorganic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R⁺, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

### Solvates

In some embodiments, the compound of formula (I) is provided in desolvated form, for example, in dehydrated form.

Alternatively, it may be convenient or desirable to prepare, purify, and/or handle a corresponding solvate of the compound.

Accordingly, in some embodiments the compound of formula (I) is provided in the form of a solvate (a complex of solute (e.g., compound, salt of compound) and solvent). Examples of solvates include hydrates, for example, a mono-hydrate, a di-hydrate and a tri-hydrate.

### Method of Synthesis

The invention provides a process for the production of a compound of formula (I) comprising the following steps:
i) treating 4-butyl-5′-methyl-2′-(prop-1-en-2-yl)-1′,2′,3′,4′-tetrahydro-[1,1′-biphenyl]-2,6-diol with an acylation reagent to produce 4-Butyl-5′-methyl-2′-(prop-1-en-2-yl)-1′,2′,3′,4′-tetrahydro-[1,1′-biphenyl]-2,6-diyl diacetate;
ii) treating 4-Butyl-5′-methyl-2′-(prop-1-en-2-yl)-1′,2′,3′,4′-tetrahydro-[1,1′-biphenyl]-2,6-diyl diacetate with an oxidising agent to produce 4-Butyl-5′-methyl-4′-oxo-2′-(prop-1-en-2-yl)-1′,2′,3′,4′-tetrahydro-[1,1′-biphenyl]-2,6-diyl diacetate; and
iii) treating 4-Butyl-5′-methyl-4′-oxo-2′-(prop-1-en-2-yl)-1′,2′,3′,4′-tetrahydro-[1,1′-biphenyl]-2,6-diyl diacetate with a reducing agent to produce the compound of Formula I.

Suitable acetylating agents include acetic anhydride, acetyl chloride, N-succinimidyl acetate, 1-acetyl-1H-1,2,3-triazolo[4,5-b]pyridine and N-acetylaimidazole. In preferred embodiments, acetic anhydride is used.

Optionally, a base is used in step i). Suitable bases include organic bases such as pyridine, dimethylbenzylamine imidazole, benzimidazole, methylimidazole, triethylamine, tributylamine, diisopropylethylamine, tetramethylethylenediamine, DABCO. In preferred embodiments, pyridine is used.

Suitable oxidising agents include chromium species such as sodium dichromate dihydrate with acetic anhydride. In preferred embodiments, acetic anhydride and sodium dichromate dihydrate are used.

Suitable reducing agents include lithium aluminium hydride, sodium bis(2-methoxyethoxy)aluminium hydride (red-AI), diborane and sodium borohydride. In preferred embodiments, lithium aluminium hydride is used.

### Intermediates

The invention provides an intermediate formed in the process of the production of a compound of formula (I), wherein the intermediate is:

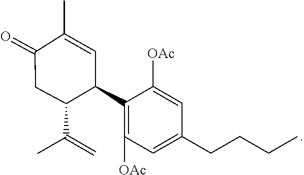 [SMILES: C=C(C)[C@@H]1CC(=O)C(C)=C[C@H]1c1c(OC(C)=O)cc(CCCC)cc1OC(C)=O]

### Pharmaceutical Compositions

While it is possible for the compound of formula (I) to be administered alone, it is preferable to administer a pharmaceutical composition (e.g., a formulation, preparation, or medicament) comprising a compound of formula (I) together with one or more other pharmaceutically acceptable ingredients.

Accordingly, the invention provides a pharmaceutical composition comprising a compound of formula (I), or a salt thereof, together with one or more pharmaceutically acceptable ingredients.

Suitable pharmaceutically acceptable ingredients (e.g. carriers, diluents, excipients, etc.) can be found in standard pharmaceutical texts, for example, Remington: The Science and Practice of Pharmacy, 20th Edition, 2000, pub. Lippincott, Williams & Wilkins; and Handbook of Pharmaceutical Excipients, 9th edition, 2020, pub. Pharmaceutical Press.

Examples of suitable pharmaceutically acceptable ingredients include pharmaceutically acceptable carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In preferred embodiments, the pharmaceutically acceptable ingredient is selected from a carrier, an oil, a disintegrant, a lubricant, a stabilizer, a flavouring agent, an antioxidant and a diluent. Optionally, another pharmaceutically effective compound may also be included.

The pharmaceutical composition may be in any suitable form. Examples of suitable forms include liquids, solutions (e.g., aqueous, nonaqueous), suspensions (e.g., aqueous, non-aqueous), emulsions (e.g., oil-in-water, water-in-oil), syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, and aerosols.

In preferred embodiments, the pharmaceutical composition may be in the form of a tablet, a capsule, a granule, a powder for inhalation, a sprinkle, an oral solution and a suspension.

### Medical Treatment

The inventors have found that compound of formula (I) is biologically active. The worked examples demonstrate that compound of formula (I) displays anticonvulsant activity in a mouse model of seizure. As such, the compound of formula (I), its salts, as well as pharmaceutical compositions comprising the compound of formula (I) or its salts, will be useful in medical treatment.

Accordingly, the invention provides a compound of formula (I), or a salt thereof, for use in a method of treatment, for example for use in a method of treatment of the human or animal body by therapy (i.e. a method of therapy).

The invention also provides a compound of formula (I), or a salt thereof, for use as a medicament.

The invention also provides a method of treatment comprising administering to a subject in need of treatment a therapeutically effective amount of a compound of formula (I), or a salt thereof.

The invention also provides the use of a compound of formula (I), or a salt thereof, for the manufacture of a medicament.

The invention also provides use of a compound of formula (I), or a salt thereof, in a method of treatment.

### Conditions Treated

The inventors have found that the compound of formula (I) displays anticonvulsant activity in a mouse model of generalised seizure. Accordingly, the compound of formula (I), its salts, as well as pharmaceutical compositions comprising the compound of formula (I) or its salts, will be useful in the treatment of certain conditions associated with seizure.

Similarly, the compounds of formula (I), its salts, as well as pharmaceutical compositions comprising the compound of formula (I) or its salts, will be useful as medicaments for treating (and in the manufacture of medicaments for treating) certain conditions associated with seizure.

In a preferred embodiment, the condition associated with seizure is epilepsy.

In one embodiment, the condition associated with seizure is generalised seizure, such as generalised seizure associated with epilepsy.

In one embodiment, the condition associated with seizure is focal-onset seizures, such as focal-onset seizure associated with epilepsy.

In one embodiment, the condition associated with seizure is tonic-clonic seizures, such as tonic-clonic seizures associated with epilepsy.

### The Subject/Patient

The method of treatment typically comprises administering a compound of formula (I), or a salt thereof, to a subject or patient.

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orang-utan, gibbon), or a human.

The subject/patient may be any of its forms of development, for example, the subject/patient may be an infant or child.

In a preferred embodiment, the subject/patient is a mammal, more preferably a human, even more preferably an adult human.

The subject/patient may also be a non-human mammal used in laboratory research, such as a rodent. Rodents include rats, mice, guinea pigs and chinchillas.

### Routes of Administration

The method of treatment may comprise administering a compound of formula (I), or a salt thereof, to a subject by any convenient route of administration, whether systemically/peripherally or topically (i.e., at the site of desired action).

The route of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection or infusion, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot or reservoir, for example, subcutaneously or intramuscularly.

### Dosages

The method of treatment typically comprises administering a therapeutically effective amount of a compound of formula (I), or a salt thereof, to a subject.

Appropriate dosages of the compound of formula (I), its salts, as well as pharmaceutical compositions comprising the compound of formula (I), or its salts, can vary from patient to patient. Determining the optimal dosage will generally involve balancing the level of therapeutic benefit against any risk or deleterious side effects. The selected dosage level will depend on a variety of factors including, but not limited to, the activity of the compound of formula (I), the route of administration, the time of administration, the rate of excretion of the compound, the duration of the treatment, other active agents, compounds, and/or materials used in combination, the severity of the condition, and the species, sex, age, weight, condition, general health, and prior medical history of the patient. The dosage and route of administration will ultimately be at the discretion of the clinician, although generally the dosage will be selected to achieve local concentrations at the site of action which achieve the desired effect without causing substantial harmful or deleterious side-effects.

In some embodiments, the dose of the compound of formula (I) is between 1 and 2,000 mg/day. In preferred embodiments, the dose is between 20 and 1,000 mg/day, more preferably between 50 and 500 mg/day.

Administration can be effected in one dose, continuously or intermittently (e.g., in divided doses at appropriate intervals) throughout the course of treatment. Single or multiple administrations can be carried out with the dose level and pattern being selected by the treating clinician.

### Co-Administration

The method of treatment may comprises administering a compound of formula (I), or a salt thereof, to a subject in the absence of other medications.

In some embodiments, the method of treatment comprises administering a compound of formula (I), or a salt thereof, to a subject in combination with one or more concomitant anti-epileptic drugs (AEDs).

Suitable AEDs include rufinamide; lamotrigine; topiramate; felbamate, stiripentol, clobazam and valproic acid.

Administration may be effected simultaneously or sequentially.

### Other Aspects and Embodiments

Each and every compatible combination of the embodiments described above is explicitly discloses herein, as if each and every combination was individually and explicitly recited.

Carious further aspects and embodiment of the present invention will be apparent to those skilled in the arti in view of the present disclosure.

Where used, “and/or” is to be taken as a specific disclosure of each of the relevant components or features alone as well as a specific disclosure of the combination of the components or features. For example, “A and/or B” is to be taken as specific disclosure of each of i) A, ii) B, and ii) A and B, just as if each were set out individually.

Unless context dictates otherwise, the descriptions and definitions of the features set out above are not limited to any particular aspect or embodiment of the invention and apply equally to all aspects ad embodiments which are described.

### Definitions

Definitions of some of the terms used to describe the invention are detailed below.

The compound described in the present application are listed below.

| Compound | Abbreviation | Short Name | Structure |
|---|---|---|---|
| Compound I | 6-OH-CBD-C4 | 6-hydroxy-cannabidiol-C4 | 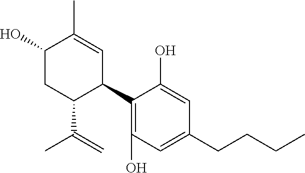 [SMILES: C=C(C)[C@@H]1C[C@H](O)C(C)=C[C@H]1c1c(O)cc(CCCC)cc1O] |

Epilepsy is considered to be a disease of the brain defined by any of the following conditions: (1) At least two unprovoked (or reflex) seizures occurring >24 h apart; (2) one unprovoked (or reflex) seizure and a probability of further seizures similar to the general recurrence risk (at least 60%) after two unprovoked seizures, occurring over the next 10 years; (3) diagnosis of an epilepsy syndrome (A practical clinical definition of epilepsy by the International League Against Epilepsy (ILAE), 2014).

The term “focal seizure” (“focal onset seizure”) refers to seizures originating within networks limited to one hemisphere. They may be discretely localized or more widely distributed. Focal seizures may originate in subcortical structures (Operational Classification of Seizure Types by the ILAE, 2017).

The term “generalized seizure” (“generalized onset seizures”) refers to seizures conceptualized as originating at some point within the brain and rapidly engaging bilaterally distributed networks (Operational Classification of Seizure Types by the ILAE, 2017).

The term “pharmaceutically acceptable” pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each ingredient (e.g. carrier, diluent, excipient, etc.) must also be “acceptable” in the sense of being compatible with the other ingredients of the composition.

The term “therapeutically-effective amount” pertains to that amount of a compound, or a material, composition or dosage form comprising a compound, which is effective for producing some desired therapeutic effect, commensurate with a reasonable benefit/risk ratio, when administered in accordance with a desired treatment regimen.

A “tonic-clonic seizure” occurs in two phases, a tonic phase typically involving muscle stiffening and loss of consciousness, and a clonic phase typically involving rhythmically jerking of the limbs.

The human dose equivalent (HED) can be estimated using the following formula:

$$H E D ( mg / kg ) = Animal dose ( mg / kg ) \times \frac{Animal {K}_{m}}{Human {K}_{m}}$$

The Kₘ for a rat is 6 and the Kₘ for a human is 37. Thus, for a human of approximately 60 Kg a 200 mg/Kg dose in rat would equate to a human daily dose of about 2,000 mg.

### Worked Examples

Certain aspects and embodiments of the invention will not be illustrated by way of example and with reference to the figures described above.

### Example 1: Synthetic Production Method for Compound I

This example describes a novel method of synthesis which was used to produce a novel compound which demonstrated pharmacological activity. Scheme 1 below describes the three stages of the reaction which was used to produce the novel compound, formed via a number of intermediates.

The novel compound of the invention is (1′R,2′R,4′S)-4-Butyl-5′-methyl-2′-(prop-1-en-2-yl)-1′,2′,3′,4′-tetrahydro-[1,1′-biphenyl]-2,4′,6-triol, and will be referred as the compound of Formula I or Compound I throughout.

Scheme 1 below shows the synthetic route of Compound I in three steps.

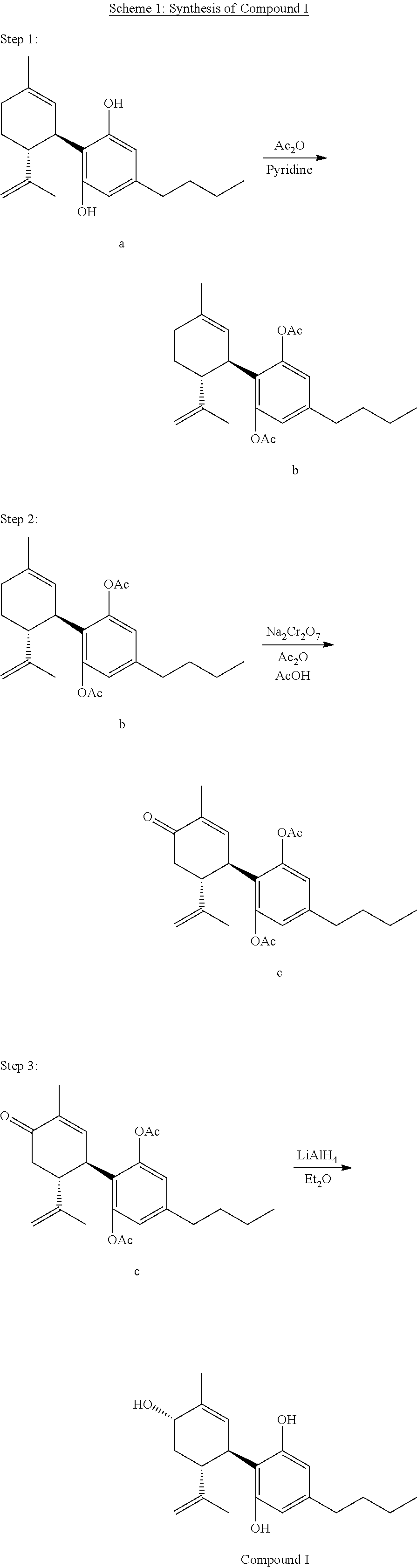

| Compound | Name |
|---|---|
| a | 4-butyl-5′-methyl-2′-(prop-1-en-2-yl)-1′,2′,3′,4′- |
| | tetrahydro-[1,1′-biphenyl]-2,6-diol |
| b | 4-Butyl-5′-methyl-2′-(prop-1-en-2-yl)-1′,2′,3′,4′- |
| | tetrahydro-[1,1′-biphenyl]-2,6-diyl diacetate |
| c | 4-Butyl-5′-methyl-4′-oxo-2′-(prop-1-en-2-yl)- |
| | 1′,2′,3′,4′-tetrahydro-[1,1′-biphenyl]-2,6-diyl diacetate |

### Step 1: Production of Compound b

A solution of (1′R,2′R)-4-butyl-5′-methyl-2′-(prop-1-en-2-yl)-1′,2′,3′,4′-tetrahydro-[1,1′-biphenyl]-2,6-diol (250 mg, 0.832 mmol) in pyridine (3.0 mL) was treated with acetic anhydride (0.24 mL, 2.5 mmol) and the reaction mixture was stirred at room temperature overnight. The mixture was diluted with ethyl acetate (80 mL) and washed with water (10 ml) and brine (10 mL). The organic layer was passed through hydrophobic filter paper and concentrated in vacuo. The residue was purified by normal phase column chromatography (40 g silica cartridge, eluting with 5-10% ethyl acetate in cyclohexane) to give the (1′R,2′R)-4-Butyl-5′-methyl-2′-(prop-1-en-2-yl)-1′,2′,3′,4′-tetrahydro-[1,1′-biphenyl]-2,6-diyl diacetate as a colourless viscous oil (236 mg, 74%).

### Step 2: Production of Compound c

A solution of (1′R,2′R)-4-Butyl-5′-methyl-2′-(prop-1-en-2-yl)-1′,2′,3′,4′-tetrahydro-[1,1′-biphenyl]-2,6-diyl diacetate (230 mg, 0.597 mmol) in acetic acid (1.0 mL) was treated with acetic anhydride (0.25 mL, 2.69 mmol) and sodium dichromate dihydrate (214 mg, 0.717) and the reaction mixture was stirred at room temperature for 3 days. The mixture was diluted with water (30 mL) and extracted with ethyl acetate (2×40 mL). The combined organic layers were washed with brine (20 mL), passed through hydrophobic filter paper and concentrated in vacuo. The residue was purified by normal phase column chromatography (25 g silica cartridge, eluting with 20-30% ethyl acetate in cyclohexane) to give (1′R,2′R)-4-Butyl-5′-methyl-4′-oxo-2′-(prop-1-en-2-yl)-1′,2′,3′,4′-tetrahydro-[1,1′-biphenyl]-2,6-diyl diacetate as a colourless oil (111 mg, 47%).

### Step 3: Production of Compound I

To a solution of lithium aluminium hydride (1.1 mL, 1.1 mmol, 1.0 M in diethyl ether) in dry diethyl ether (3 mL) cooled to 0° C. was added dropwise a solution of (1′R,2′R)-4-butyl-5′-methyl-4′-oxo-2′-(prop-1-en-2-yl)-1′,2′,3′,4′-tetrahydro-[1,1′-biphenyl]-2,6-diyl diacetate (105 mg, 0.263 mmol) in diethyl ether (3.0 mL). The reaction mixture was warmed to room temperature and stirred for 4 hours. The mixture was re-cooled to 0° C. and water (1 mL) was added cautiously followed by 2 M hydrochloric acid (5 mL). The mixture was extracted with ethyl acetate (50 mL) and the organic layer was washed with brine (20 mL), passed through hydrophobic filter paper and concentrated in vacuo. The residue was purified by normal phase column chromatography (12 g silica cartridge, eluting with 10-50% ethyl acetate in cyclohexane) followed by reverse phase preparative HPLC to give Compound I as an off-white solid (23 mg, 28%).

### Example 2: Evaluation of Novel Compounds for Anticonvulsant Activity Using the Maximal Electroshock Seizure Threshold (MEST) Test in the Mouse Using Minimal Sample Size (Mini-MEST)

The efficacy of the novel compound according to Formula I was tested in a novel mouse model of generalised seizure, the mini-MEST (maximal electroshock seizure threshold) test, which uses lower n numbers than typically used.

The maximal electroshock seizure threshold (MEST) test is widely utilized preclinically to evaluate pro- or anti-convulsant properties of test compounds (Loscher et al., 1991).

In the MEST test the ability of a drug to alter the seizure threshold current required to induce hind limb tonic extensor convulsions is measured according to an “up and down” method of shock titration (Kimball et al., 1957). An increase in seizure threshold is indicative of anti-convulsant effect. Antiepileptic drugs including the sodium channel blockers (e.g. lamotrigine) with clinically proven efficacy against generalised tonic-clonic seizures all exhibit anti-convulsant properties in this test in the mouse.

Conversely, a reduction in seizure threshold is indicative of a pro-convulsant effect as observed with known convulsant agents such as picrotoxin.

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC₅₀) determines the seizure threshold for the treatment group and the effects were then compared to the CC₅₀ of the vehicle control group.

### Methods

### Study Details:

Naïve mice were acclimatized to the procedure room in their home cages for up to 7 days, with food and water available ad libitum.

All animals were weighed at the beginning of the study and randomly assigned to treatment groups based on a mean distribution of body weight across groups. All animals were dosed at 10 mL/kg via intraperitoneal (i.p) injection, with either vehicle, test compound at 100 mg/kg (Compound I) or 150 mg/kg (Compound II), or diazepam at 2.5 mg/kg.

Animals were individually assessed for the production of a tonic hind limb extensor convulsion at 30 min post-dose for vehicle, test compound and diazepam, from a single electroshock.

The first animal within a treatment group was given a shock at the expected or estimated CC₅₀ current. For subsequent animals, the current was lowered or raised depending on the convulsions outcome from the preceding animal in log scale intervals.

Data generated from each treatment group were used to calculate the CC₅₀±SEM values for the treatment group.

### Test Compounds:

Vehicle: (5% ethanol, 10% solutol in 85% Saline) was prepared as follows: 1 mL of ethanol, 2 mL of solutol were warmed to 60° C., in 17 mL of saline (1:2:17).
Positive control: diazepam was used at 2.5 mg/kg.

The test compound, described herein as Compound I, are as shown as Formula I. Test compound was administered at 150 mg/kg (i.p.) in a 1:2:17 ethanol:solutol:0.9% saline formulation.

### Sample Collection:

Each animal was humanely killed immediately after production of a convulsion by destruction of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4° C. for 10 minutes at 1500×g. The resulting plasma was removed (>100 μL) and split into 2 aliquots of 0.5 mL Eppendorf tubes containing 10 μL of ascorbic acid (100 mg/mL) for stabilisation. Brains were removed, washed in saline and halved. Each half was placed into separate 2 mL screw cap cryovials, weighed and frozen on cardice.

### Statistical Analysis

The data for each treatment group were recorded as the number of +'s and 0's at each current level employed and this information is then used to calculate the CC₅₀ value (current required for 50% of the animals to show seizure behaviour)±standard error.

Test compound effects were also calculated as percentage change in CC₅₀ from the vehicle control group.

Significant difference between drug-treated animals and controls were assessed according to Litchfield and Wilcoxon (1949).

### Results

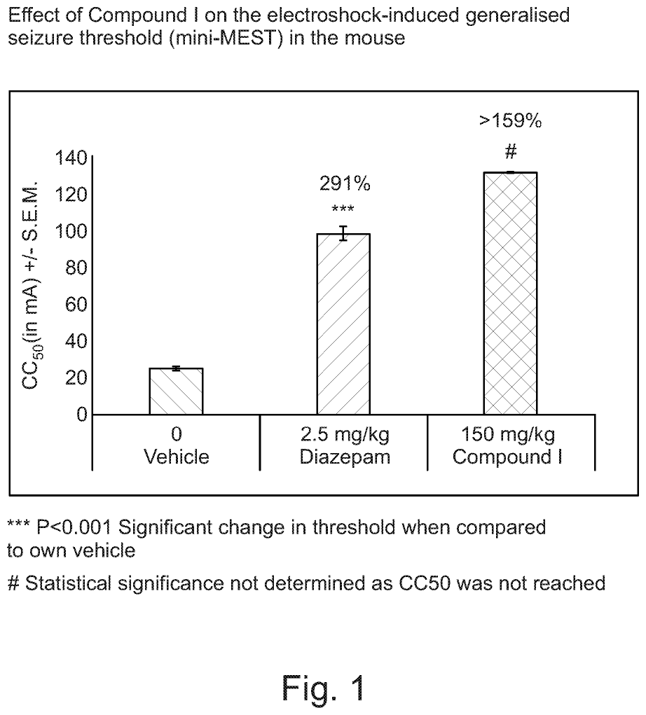

FIG. 1 and Table 1 describe the data produced in this experiment.

In the vehicle group, the CC₅₀ value was calculated to be 25.0 mA.

In the diazepam (2.5 mg/kg) treated group, administered i.p. 30 minutes before the test, the CC₅₀ value was 97.8 mA. This result was statistically significant (p<0.001) compared to vehicle control.

Compound I, administered i.p. 30 minutes before the test, produced a clear increase in seizure threshold as compared to vehicle, with CC₅₀>131 mA for 150 mg/kg; an exact value was not calculated as a “+” tonic hindlimb convulsion was not seen within the 6 animals tested. Although CC₅₀ was not determined, Compound I showed a clear increase in seizure threshold in the mini-MEST. Clear activity of Compound I was demonstrated as animals in this treatment group did not have any convulsions.

Such data are indicative that this compound will be of therapeutic benefit.

TABLE 1
Evaluation of effect of Compound I in the mini-MEST test

| | | Test time | | | | % change |
|---|---|---|---|---|---|---|
| | Dose | post dose | | CC₅₀ +/− | Signif- | from |
| Treatment | (mg/kg) | (min) | N | SEM | icance | vehicle |
| Vehicle | — | 30 | 6 | 25.0 ± 1.1 | — | — |
| Diazepam | 2.5 | 30 | 6 | 97.8 ± 3.7 | P < | 291% |
| | | | | | 0.001 | |
| Compound I | 150 | 30 | 6 | >131 | # | >159% |

# Statistical significance not determined as CC₅₀ was not reached

### Conclusions

This data produced using the mini-MEST model demonstrates a therapeutic effect for this compound. Compound I demonstrated a strong therapeutic effect, with CC₅₀ increasing by more than 159% compared to vehicle control.

This data is significant as it provides heretofore unknown evidence that this novel compound may be of therapeutic value.

### Example 3: Bioanalysis of Novel Compound

Bioanalysis experiments were carried out to determine the quantitative measurement of Compound I in plasma and brain.

### Methods

Six mice were dosed with Compound I at 150 mg/kg via intraperitoneal (i.p) injection. Sample analysis was performed 30 min post-dose using a sample volume of 40 μL. Following addition of internal standard (in acetonitrile) and Isopropanol samples were protein precipitated by addition of acetonitrile. Samples were centrifuged and supernatant transferred to a clean 96-well plate (with addition of Tween to minimise non-specific binding to plate materials), evaporated to dryness then reconstituted in suitable solvent. The extracts quantified by ultraperformance liquid chromatography-tandem mass spectromtry (UPLC-MS/MS).

### Results

Table 2 shows the data produced in this experiment. High concentrations of Compound I was detected in both plasma and brain.

TABLE 2
Concentrations of Compound I

| | Dose | | Plasma Conc. | Mean Plasma | |
|---|---|---|---|---|---|
| Treatment | (mg/kg) | Location | (nM) | Conc. (nM) | SD |
| Compound | 150 | Plasma | 47322 | 255449 | 111909 |
| I | | | 295516 | | |
| | | | 268440 | | |
| | | | 299404 | | |
| | | | 379642 | | |
| | | | 242369 | | |
| Compound | 150 | Brain | 17696 | 169460 | 76867 |
| I | | | 200472 | | |
| | | | 183889 | | |
| | | | 173842 | | |
| | | | 212516 | | |
| | | | 228344 | | |

### Conclusions

This data confirms presence of Compound I in plasma and brain

1. A compound of formula (I), or a salt thereof:

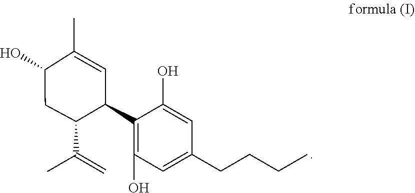 [SMILES: C=C(C)[C@@H]1C[C@H](O)C(C)=C[C@H]1c1c(O)cc(CCCC)cc1O]

2. The compound of claim 1 as a pure, isolated or synthetic compound.

3. A pharmaceutical composition comprising a compound of formula (I) or a salt thereof.

4. The pharmaceutical composition of claim 3 in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

5. The pharmaceutical composition of claim 3 or claim 4 comprising one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

6. A compound of formula (I), or a salt thereof, for use a medicament.

7. The compound for use of claim 6, wherein the medicament is a medicament for treating epilepsy.

8. The compound for use of claim 6 or claim 7, wherein the medicament is a medicament for treating generalised seizures, focal-onset seizures, or tonic-clonic seizures.

9. A compound of formula (I), or a salt thereof, for use in a method of treatment.

10. The compound for use of claim 9, wherein the method of treatment is a method of treating epilepsy.

11. The compound for use of claim 9 or claim 10, wherein the method of treatment is a method of treating generalised seizures, focal-onset seizures, or tonic-clonic seizures.

12. The compound for use of any of claims 9 to 11, wherein the compound is used in combination with one or more concomitant anti-epileptic drugs (AEDs).

13. The compound for use of claim 12, wherein the AEDs are selected from rufinamide; lamotrigine; topiramate; felbamate, stiripentol, clobazam and valproic acid.

14. The compound for use of any of claims 9 to 13, wherein the dose of the compound is between 1 and 2,000 mg/day, such as between 20 and 1,000 mg/day, such as between 50 and 500 mg/day.

15. A method of treatment comprising administering to a subject in need of treatment a therapeutically effective amount of a compound of formula (I) or a salt thereof.

16. The method of treatment of claim 15, wherein the method of treatment is a method of treating epilepsy.

17. The method of treatment of claim 15 or 16, wherein the method of treatment is a method of treating generalised seizures, focal-onset seizures, or tonic-clonic seizures.

18. A process for the production of a compound of formula (I) comprising the following steps:
i) treating 4-butyl-5′-methyl-2′-(prop-1-en-2-yl)-1′,2′,3′,4′-tetrahydro-[1,1′-biphenyl]-2,6-diol with an acylation reagent to produce 4-Butyl-5′-methyl-2′-(prop-1-en-2-yl)-1′,2′,3′,4′-tetrahydro-[1,1′-biphenyl]-2,6-diyl diacetate;
ii) treating 4-Butyl-5′-methyl-2′-(prop-1-en-2-yl)-1′,2′,3′,4′-tetrahydro-[1,1′-biphenyl]-2,6-diyl diacetate with an oxidising agent to produce 4-Butyl-5′-methyl-4′-oxo-2′-(prop-1-en-2-yl)-1′,2′,3′,4′-tetrahydro-[1,1′-biphenyl]-2,6-diyl diacetate; and
iii) treating 4-Butyl-5′-methyl-4′-oxo-2′-(prop-1-en-2-yl)-1′,2′,3′,4′-tetrahydro-[1,1′-biphenyl]-2,6-diyl diacetate with a reducing agent to produce the compound of Formula I.

19. The method of claim 18, wherein the acylation reagent is selected from acetic anhydride, acetyl chloride, N-succinimidyl acetate, 1-acetyl-1 H-1,2,3-triazolo[4,5-b]pyridine and N-acetylaimidazole; such as acetic anhydride.

20. The method of claim 18 or 19, wherein the base is selected from pyridine, dimethylbenzylamine imidazole, benzimidazole, methylimidazole, triethylamine, tributylamine, diisopropylethylamine, tetramethylethylenediamine, DABCO; such as pyridine.

21. The method of any of claims 18 to 20, wherein the oxidising agent is a chromium oxidising agent, such acetic anhydride and sodium dichromate dihydrate.

22. The method of any of claims 18 to 21, wherein the reducing agent is selected from lithium aluminium hydride, sodium bis(2-methoxyethoxy)aluminium hydride (red-AI), diborane and sodium borohydride; such as lithium aluminium hydride.

23. An intermediate formed in the process of the production of a compound of formula (I), wherein the intermediate is selected from: 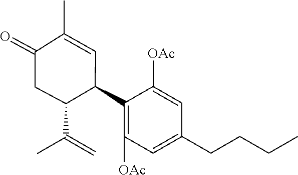 [SMILES: C=C(C)[C@@H]1CC(=O)C(C)=C[C@H]1c1c(OC(C)=O)cc(CCCC)cc1OC(C)=O]
